(19) United States
(12) Patent Application Publication
(10) Pub. No.: US 2026/0096981 A1
(43) Pub. Date: Apr. 9, 2026

(54) COSMETIC COMPOSITION

(71) Applicant: LaLa Co., Ltd, Jeju-si (KR)

(72) Inventor: Young Hee KIM, Jeju-si (KR)

(73) Assignee: LaLa Co., Ltd, Jeju-si (KR)

(21) Appl. No.: 18/908,548

(22) Filed: Oct. 7, 2024

(51) Int. Cl. A61K 8/9789; A61K 8/29; A61K 8/9711; A61Q 17/00; A61Q 17/04

(52) U.S. Cl. CPC A61K 8/9789; A61K 8/29; A61K 8/9711; A61Q 17/005; A61Q 17/04

(57) ABSTRACT

Proposed is a cosmetic composition containing an extract of a mixture of citrus peel and Sargassum muticum and titanium dioxide as active ingredients.

### BACKGROUND OF THE DISCLOSURE

### 1. Field of the Disclosure

The present disclosure relates to a cosmetic composition containing an extract of a mixture of citrus peel and Sargassum muticum and titanium dioxide as active ingredients.

### 2. Description of the Related Art

In modern society, there has recently been a significantly growing interest in skin health due to environmental pollution and abrupt climate change. In particular, skin damage caused by ultraviolet (UV) rays is responsible for aging, melasma, blemishes, and the like, so the need for UV protection has been emphasized among consumers. Accordingly, active development of various UV protection products (UV protection cosmetics) capable of protecting the skin from UV rays is being conducted.

In the meantime, protecting the skin from fine dust, bacteria, and other pollutants is also emerging as an important skin care factor. Bacteria that grow on the surface of the skin can cause various skin breakouts, including acne and inflammation. Accordingly, the demand for products with antimicrobial functions to control such skin problems is steadily growing.

However, cosmetics currently available in the market offer either a UV protection or antimicrobial function separately in most cases, and only a few provide both functions simultaneously. As a result, consumers separately use sunscreens for UV protection and another for antimicrobial care, which is inconvenient.

Therefore, there is a need to develop cosmetic compositions that can protect the skin from UV rays while preventing skin problems caused by bacteria. In particular, the need for products that can provide such complex functions while being gentle on the skin and maintaining stable effects over a long period of time has been growing.

### DOCUMENT OF RELATED ART

### Patent Document

Korean Patent No. 10-2142311

### SUMMARY OF THE INVENTION

The present disclosure aims to provide a cosmetic composition that satisfies both antimicrobial activity and UV protection ability, using an extract of a mixture of citrus peel and Sargassum muticum and titanium dioxide. Other objectives of the present disclosure will be presented below.

As will be confirmed in the examples and experimental examples below, an extract of a mixture of citrus peel and Sargassum muticum shows antimicrobial activity in the present disclosure. In particular, it is seen that a much better effect is demonstrated in the case of using the extract of the mixture than in the case of each independently using a citrus peel extract and a Sargassum muticum extract.

A cosmetic composition of the present disclosure, which is provided on the basis of such experimental results, is characterized by including the extract of the mixture of citrus peel and Sargassum muticum and titanium dioxide as active ingredients.

As used herein, the term “extract” refers to an extract obtained by leaching an object to be extracted using water, lower alcohols having 1 to 4 carbon atoms, such as methanol, ethanol, and butanol, methylene chloride, ethylene, acetone, hexane, ether, chloroform, ethyl acetate, butyl acetate, N,N-dimethylformamide (DMF), dimethylsulfoxide (DMSO), 1,3-butylene glycol, propylene glycol, or a mixture of the abovementioned solvents, an extract obtained using a supercritical extraction solvent, such as carbon dioxide and pentane, or a fraction obtained by fractionating the extract. In addition, considering the polarity of active materials, degree of extraction, and degree of preservation, any methods such as cold dipping, reflux, heating, ultrasonic irradiation, supercritical extraction, and the like may be used as the extraction method. What is referred to as the extract obtained through fractioning includes: fractions obtained by suspending the extract in a predetermined solvent, mixing the suspended extracted with a solvent that differs in polarity, and then leaving the resulting mixture to stand still; and fractions obtained by adsorbing the extract onto a column filled with silica gel and the like and then using a hydrophobic solvent, a hydrophilic solvent, or a mixture of the hydrophobic and hydrophilic solvents as the mobile phase. In addition, what is referred to as the extract includes a concentrated liquid extract or solid extract from which the extraction solvent is removed by methods such as freeze drying, vacuum drying, hot air drying, spray drying, and the like. The extract preferably refers to an extract obtained using water, ethanol, or a mixture of water and ethanol as an extraction solvent and, more preferably, refers to an extract obtained using a mixture of water and ethanol as an extraction solvent.

In addition, as used herein, the term “active ingredient” refers to a component capable of showing the desired activity when used alone or in conjunction with a carrier that is inactive on its own.

In order to enhance or add skin-related activities, the cosmetic composition of the present disclosure may be prepared in a form containing skin-whitening ingredients, anti-wrinkle ingredients, skin-moisturizing ingredients, and the like known in the art, in addition to the active ingredients. Specifically, examples of the skin-whitening ingredients may include arbutin, niacinamide, ascorbyl glucoside, alpha-bisabolol, oil-soluble licorice (glycyrrhiza) extract, and the like. Examples of the anti-wrinkle ingredients may include retinol, retinyl palmitate, adenosine, polyethoxylated amide, and the like. Examples of the skin-moisturizing ingredients may include AP collagen enzyme-degrading peptide, Collactive collagen peptide, N-acetylglucosamine, konjac potato extract, complex extract of dandelion and the like, rice bran extract, corn germ extract, low-molecular-weight collagen peptides, licorice extract powder, phosphatidylserine, and hyaluronic acid.

In the cosmetic composition of the present disclosure, the active ingredient may be contained in any amount within a range that is not toxic to the human body, depending on the use, formulation, and the like. Specifically, the active ingredient may be contained in an amount in the range of 0.001 to 99.99 wt %.

The cosmetic composition of the present disclosure, depending on the use thereof, may take on various product classifications from a legal standpoint. Specifically, the cosmetic composition may be a functional cosmetic for skin-whitening use, a non-functional common cosmetic, or the like. The cosmetic composition may also take on various forms in terms of formulations. Specifically, the cosmetic composition may be formulated as a solution, suspension, emulsion, paste, gel, cream, lotion, powder, soap, surfactant-containing cleanser, oil, powder foundation, emulsion foundation, wax foundation, spray, or the like. Specifically, the cosmetic composition may be formulated as a softening toner, nourishing toner, nourishing cream, massage cream, essence, eye cream, cleansing cream, cleansing foam, cleansing water, facial mask, spray, powder, or the like.

When formulated as a paste, cream, or gel in the present disclosure, an animal oil, vegetable oil, wax, paraffin, starch, tragacanth, cellulose derivative, polyethylene glycol, silicone, bentonite, silica, talc, zinc oxide, or the like may be used as a carrier component.

When formulated as a powder or spray in the present disclosure, lactose, talc, silica, aluminum hydroxide, calcium silicate, or polyamide powder may be used as the carrier component. In particular, when formulated as a spray, a propellant, such as chlorofluorohydrocarbon, propane/butane, or dimethyl ether, may be additionally contained.

When formulated as a solution or emulsion in the present disclosure, a solvent, solubilizing agent, or emulsifier may be used as the carrier component, and examples thereof used may, for example, include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When formulated as a suspension in the present disclosure, a liquid diluent, such as water, ethanol, or propylene glycol, a suspending agent, such as ethoxylated isostearyl alcohol, polyoxyethylene sorbitol ester, or polyoxyethylene sorbitan ester, microcrystalline cellulose, aluminum metahydroxide, bentonite, agar, or the like may be used as the carrier component.

When formulated as a surfactant-containing cleanser in the present disclosure, aliphatic alcohol sulfate, aliphatic alcohol ether sulfate, sulfosuccinic acid monoester, isethionate, an imidazolinium derivative, methyl taurate, sarcosinate, fatty acid amide ether sulfate, alkylamidobetaine, aliphatic alcohol, fatty acid glyceride, fatty acid diethanolamide, vegetable oil, a lanolin derivative, ethoxylated glycerol fatty acid ester, or the like may be used as the carrier component.

As described above, according to the present disclosure, a cosmetic composition with antimicrobial activity and UV protection ability can be provided using an extract of a mixture of citrus peel and Sargassum muticum and titanium dioxide.

According to the present disclosure, a cosmetic composition capable of controlling phototoxicity while demonstrating a uniform UV protection effect can also be provided.

### DESCRIPTION OF THE PREFERRED EMBODIMENTS

Hereinafter, the present disclosure will be described with reference to examples and experimental examples. However, the scope of the present disclosure is not limited to the examples and experimental examples.

The present disclosure relates to a cosmetic composition containing an extract of a mixture of citrus peel and Sargassum muticum and titanium dioxide as active ingredients.

Citrus peel is rich in natural antimicrobial substances such as flavonoids, limonene, hesperidin, and the like, and thus has antimicrobial and anti-inflammatory effects.

In addition, Sargassum muticum, a seaweed that grows in the ocean, contains unique antimicrobial ingredients and antioxidant substances and thus is effective in inhibiting the growth of bacteria and fungi.

In particular, the present disclosure relates to the extract of the mixture of citrus peel and Sargassum muticum, which allows the antimicrobial effect to be multiplied compared to that in the case of each independently using a citrus peel extract and a Sargassum muticum extract, as shown in the following experimental examples.

This is determined to be achieved because the flavonoids in the citrus peel extract and the polysaccharides, mineral ingredients, and the like in the Sargassum muticum extract can function together and increase antimicrobial activity.

In the case of each independently using the citrus peel extract and the Sargassum muticum extract for a long period of time, microorganisms are likely to develop resistance to the ingredients therein. However, in the case of the extract of the mixture, as in the present disclosure, the risk of resistance development is lowered because each ingredient has a different antimicrobial mechanism, enabling the antimicrobial effect to last longer.

It is appropriate that the mixture of citrus peel and Sargassum muticum is a mixture in which citrus peel and Sargassum muticum are mixed in a weight ratio in the range of 40:60 to 60:40.

The present disclosure also provides one example in which the extract of the mixture of citrus peel and Sargassum muticum further contains titanium dioxide.

Preferably, it is appropriate to mix 5 to 15 parts by weight of titanium dioxide with respect to 100 parts by weight of the extract of the mixture.

Titanium dioxide mentioned above is an effective ingredient in UV protection and serves to protect the skin by blocking both ultraviolet A (UVA) and ultraviolet B (UVB) rays.

The present disclosure also provides one example in which ethylene glycol is further added, as well as titanium dioxide.

Typically, titanium dioxide is characterized in that uniform dispersion thereof in a composition is challenging. In order to improve such properties in the present disclosure, ethylene glycol is added to induce uniform dispersion of titanium dioxide, which allows the UV protection function to be demonstrated uniformly throughout the skin.

Furthermore, when ethylene glycol is added, the surface of the titanium dioxide particles is substituted with hydroxyl groups (—OH), and the absolute zeta potential value becomes high, thus improving dispersibility. When the alkali concentration is high during such a process, the substitution amount of hydroxyl groups (—OH) increases, which controls photocatalytic activity.

In other words, the addition of ethylene glycol allows uniform UV protection ability to be demonstrated while enabling phototoxicity control by controlling the photocatalytic activity.

Preferably, it is appropriate to mix 0.1 to 2 parts by weight of ethylene glycol with respect to 100 parts by weight of the extract of the mixture.

In addition, the present disclosure provides one example in which the extract of the mixture of citrus peel and Sargassum muticum further contains a purple cabbage extract to multiply the phototoxicity control efficiency of titanium dioxide.

In other words, the purple cabbage extract is allowed to be further contained to multiply the phototoxicity inhibition ability of titanium dioxide while providing a safe UV protection effect. In addition, purple cabbage extract, a component with a strong antioxidant effect, enhances a skin protection function and helps maintain the stability of UV protection components when used in conjunction with titanium dioxide.

Preferably, it is appropriate to mix 3 to 8 parts by weight of the purple cabbage extract with respect to 100 parts by weight of the extract of the mixture.

Hereinafter, preferred embodiments of the present disclosure will be described by the following experimental examples.

### <Comparative Example 1> Preparation of Citrus Peel Extract

To 30 g of citrus peel powder, 70% ethanol in an amount corresponding to 10 times as much the weight of the citrus peel powder was added. Then, the resulting product was extracted at room temperature for 24 hours and filtered through filter paper. The resulting filtrate was concentrated under reduced pressure and freeze-dried to obtain a citrus peel extract.

### <Comparative Example 2> Preparation of Sargassum muticum Extract

To 30 g of Sargassum muticum powder, 70% ethanol in an amount corresponding to 10 times as much the weight of the Sargassum muticum powder was added. Then, the resulting product was extracted at room temperature for 24 hours and filtered through filter paper. The resulting filtrate was concentrated under reduced pressure and freeze-dried to obtain a Sargassum muticum extract.

### <Example 1> Preparation of Extract of Mixture of Citrus Peel and Sargassum muticum

To 30 g of citrus peel powder and 30 g of Sargassum muticum powder, 70% ethanol in an amount corresponding to 10 times as much the weight of the powder mixture was added. Then, the resulting product was extracted at room temperature for 24 hours and filtered through filter paper. The resulting filtrate was concentrated under reduced pressure and freeze-dried to obtain an extract of the mixture of citrus peel and Sargassum muticum.

### Example 2

A sample was prepared by mixing 10 parts by weight of titanium dioxide (with a particle size of 2 μm) with respect to 100 parts by weight of the extract prepared by Example 1.

### Example 3

A sample was prepared in the same manner as in Example 2, except for mixing 1 part by weight of ethylene glycol with respect to 100 parts by weight of the extract.

### Example 4

A sample was prepared in the same manner as in Example 3, except for mixing 5 parts by weight of a purple cabbage extract with respect to 100 parts by weight of the extract. In this case, the purple cabbage extract was obtained by the following processes: to 30 g of purple cabbage powder, adding 70% ethanol in an amount corresponding to 10 times as much the weight of the purple cabbage powder, extracting the resulting mixture at room temperature for 24 hours, filtering the extract through filter paper, concentrating the resulting filtrate under reduced pressure, and freeze-drying the concentrated filtrate.

### <Evaluation of Antimicrobial Activity>

Evaluation of antimicrobial activity was performed on each sample of Comparative Examples 1 and 2 and Examples 1 to 4, using a handprinting kit for antimicrobial evaluation. Two handprinting kits for antimicrobial evaluation were each independently prepared for acne bacteria (Propionibacterium acnes) and general bacteria. One kit was brought into contact with unwashed hands, and the other was brought into contact with hands washed by spraying 10 ml of each sample. Then, the number of colonies developed after 24 hours was counted. The results thereof are shown in Table 1 below.

TABLE 1
(TNTC: too numerous to count)

| | Kit for acne bacteria | | Kit for general bacteria | |
|---|---|---|---|---|
| | Before use | After use | Before use | After use |
| Comparative | 117 ± 5 | 92 ± 5 | TNTC | 111 ± 5 |
| Example 1 | | | | |
| Comparative | 119 ± 5 | 87 ± 5 | TNTC | 109 ± 5 |
| Example 2 | | | | |
| Example 1 | 122 ± 5 | 38 ± 5 | TNTC | 63 ± 5 |
| Example 2 | 115 ± 5 | 43 ± 5 | TNTC | 67 ± 5 |
| Example 3 | 117 ± 5 | 35 ± 5 | TNTC | 58 ± 5 |
| Example 4 | 121 ± 5 | 44 ± 5 | TNTC | 70 ± 5 |

As shown in Table 1 above, it is seen that antimicrobial activities in the examples are better than those in the comparative examples. As shown in the above experiments, it is seen that a much better effect is demonstrated in terms of antimicrobial activities in the case of using the extract of the mixture of citrus peel and Sargassum muticum than in the case of each independently using the citrus peel extract and the Sargassum muticum extract. It is also seen that in the case of Examples 1 to 4, almost similar antimicrobial activities are demonstrated. This indicates that the antimicrobial activity of the extract of the mixture of citrus peel and Sargassum muticum is not impaired even when further adding titanium dioxide or the like to Examples 2 to 4.

### <Experiment of UV Protection Ability>

As shown in Table 2 below, UV protection products of Examples 2-2 to 4-4 were prepared by adding the samples of Examples 2 to 4, respectively. A basic formulation of the UV protection products was prepared using the following components based on a sun protection factor (SPF) of 50. To prepare a UV protection cream, water-soluble ingredients such as purified water, glycerin, and the like were mixed in one water-based beaker and heated to 75° C. In another oil-based beaker, a surfactant, oil-soluble ingredients such as cyclohexasiloxane and the like were contained, mixed, and heated to 75° C. The water-based and oil-based beakers were mixed using a homomixer at a speed of 3700 rpm for 5 minutes. Then, the resulting mixture was mixed using a paddle mixer, cooled, and mixed with a preservative and each sample of Examples 2 to 4 at a temperature of 50° C. or lower, thus completing the preparation of the UV protection product.

TABLE 2

| | Content (%) | | |
|---|---|---|---|
| Ingredients | Example 2-2 | Example 3-3 | Example 4-4 |
| Purified water | To 100 | To 100 | To 100 |
| Glycerin | 5.0 | 5.0 | 5.0 |
| Butylene glycol | 3.0 | 3.0 | 3.0 |
| Ethylhexyl methoxycinnamate | 4.0 | 4.0 | 4.0 |
| Ethylhexyl palmitate | 5.0 | 5.0 | 5.0 |
| Cyclopentasiloxane | 4.0 | 4.0 | 4.0 |
| Ethylhexyl salicylate | 2.0 | 2.0 | 2.0 |
| Octocrylene | 1.0 | 1.0 | 1.0 |
| Cyclohexasiloxane | 0.8 | 0.8 | 0.8 |
| Glycol stearate | 0.2 | 0.2 | 0.2 |
| Glyceryl stearate | 0.2 | 0.2 | 0.2 |
| Triethanolamine | 0.1 | 0.1 | 0.1 |
| Propylparaben | 0.1 | 0.1 | 0.1 |
| Example 2 | 4.0 | | |
| Example 3 | | 4.0 | |
| Example 4 | | | 4.0 |

As shown in Table 2 above, UV protection effects were tested using the UV protection products of Examples 2-2 to 44. The UV protection effect was measured using an SPF-290S analyzer by a measurement method in which 2 mg/cm² of the sample in each example was applied on a Transpore tape (3M) and then dried for 30 minutes. The results thereof are shown in Table 3.

TABLE 3

| | Example 2-2 | Example 3-3 | Example 4-4 |
|---|---|---|---|
| SPF | 21 | 38 | 37 |

As shown in Table 3, it is seen that the SPF value is higher in the case of Examples 3-3 and 4-4 than in the case of Example 2-2, which is determined that the induction of uniform dispersion of titanium dioxide is attributed to ethylene glycol further added to Examples 3-3 and 4-4.

### <Experiment of Phototoxicity>

Skin patch tests were conducted on 15 members of the public using the UV protection products prepared above to test skin irritation. The results thereof are shown in Table 4.

TABLE 4

| Classification | Example 2-2 | Example 3-3 | Example 4-4 |
|---|---|---|---|
| +++ | 1 | 1 | 0 |
| ++ | 7 | 4 | 1 |
| + | 3 | 3 | 2 |
| ± | 2 | 6 | 11 |
| − | 2 | 1 | 1 |

+++: severe skin breakouts, ++: mild skin breakouts, +: minor skin breakouts, ±: normal, −: negative

As shown in Table 4 above, when comparing Example 2-2 with Example 3-3, the respective numbers in the case with mild skin breakouts appear to be 7 and 4, and the respective numbers in the normal case appear to be 2 and 6, which is determined that the reduction of photocatalytic activity is attributed to ethylene glycol further added to Example 3-3. When comparing Example 3-3 with Example 4-4, the respective numbers in the case with mild skin breakouts appear to be 4 and 1, and the respective numbers in the normal case appear to be 6 and 11, which is determined that the control of the phototoxicity of titanium dioxide is attributed to the purple cabbage extract further added to Example 4-4.

What is claimed is:

1. A cosmetic composition comprising:
an extract of a mixture of a citrus peel and Sargassum muticum; and
titanium dioxide, wherein both the extract and the titanium dioxide serve as active ingredients.

2. The cosmetic composition of claim 1, further comprising ethylene glycol.

3. The cosmetic composition of claim 2, further comprising a purple cabbage extract.

4. The cosmetic composition of claim 1, wherein the composition is for antimicrobial use against acne bacteria.

5. The cosmetic composition of claim 1, wherein the composition is for use in ultraviolet (UV) protection.

6. The cosmetic composition of claim 2, wherein the composition is for antimicrobial use against acne bacteria.

7. The cosmetic composition of claim 3, wherein the composition is for antimicrobial use against acne bacteria.

8. The cosmetic composition of claim 2, wherein the composition is for use in ultraviolet (UV) protection.

9. The cosmetic composition of claim 3, wherein the composition is for use in ultraviolet (UV) protection.
